(19) United States
(12) Patent Application Publication
(10) Pub. No.: US 2026/0102589 A1
(43) Pub. Date: Apr. 16, 2026

(54) TUBING CLIP AND METHOD OF USING SAME

(71) Applicant: ORLANDO HEALTH, INC., Orlando, FL (US)

(72) Inventors: Matthew SOLAR, Orlando, FL (US); Evan Cuellar GOMEZ, Orlando, FL (US); Raeanna Lee WHITE, Orlando, FL (US); Jennifer Lynne JONES, Orlando, FL (US)

(73) Assignee: ORLANDO HEALTH, INC., Orlando, FL (US)

(21) Appl. No.: 18/915,719

(22) Filed: Oct. 15, 2024

(51) Int. Cl. A61M 25/02

(52) U.S. Cl. CPC A61M 25/02; A61M 2025/024

(57) ABSTRACT

A clip for securing a tubing, including: a base portion disposed between a tab portion and a wall portion, where the base portion includes at least one rib. The tab portion is resiliently biased toward a first orientation and configured to be twisted to a second orientation. The tab portion is configured to be releasably secured to the wall portion, and the clip is movable between a locked configuration and an unlocked configuration. In the locked configuration, a tubular space is formed between surfaces of the base portion, the tab portion, and the wall portion, and the tubular space is configured to secure at least a portion of a tubing therein.

### TECHNICAL FIELD

The present disclosure relates generally to a device/method for securing a tubing.

### BACKGROUND

Various kinds of tubing are used in numerous settings across many different industries. For example, in medical procedures (e.g., external ventricular drainage (EVD)), tubing with different diameters may be used for different purposes, and it is important to be able to secure the tubing (e.g., on the patient), as desired and/or needed.

### SUMMARY

One general aspect of the present disclosure includes a clip for securing a tubing, including: a base portion disposed between a tab portion and a wall portion, where the base portion includes at least one rib, where the tab portion is resiliently biased toward a first orientation and configured to be twisted to a second orientation, where the wall portion is configured to releasably secure the tab portion, where the clip is movable between a locked configuration and an unlocked configuration, where the tab portion includes a first locking structure, the wall portion includes a through opening and a second locking structure, and the first locking structure is configured to extend through the through opening when in the second orientation (but not in the first orientation) and to releasably engage the second locking structure of the wall portion, such that the tab portion is secured to the wall portion and the clip is in the locked configuration when in the first orientation, and where, in the locked configuration, a tubing-securing space is formed among the base portion, the tab portion, and the wall portion, and the at least one rib protrudes into the tubing-securing space.

Another general aspect of the present disclosure includes a clip for securing a tubing, including: a base portion extending between a first end and a second end; a wall portion extending upwardly from the first end of the base portion; a tab portion extending upwardly from the second end of the base portion, where the tab portion is resiliently biased toward a first orientation and configured to be twisted to a second orientation, where the clip is movable between a locked configuration and an unlocked configuration, and in the locked configuration, a tubing-securing opening is formed by inward facing surfaces of the base portion, the tab portion, and the wall portion, where the tab portion includes a first locking structure, and the first locking structure has a length dimension and a width dimension, where in the unlocked configuration and in the first orientation, the length dimension of the first locking structure extends substantially perpendicular to a longitudinal axis of the tubing-securing space, where in the unlocked configuration and in the second orientation, the length dimension of the first locking structure extends substantially parallel to the longitudinal axis of the tubing-securing space, where the wall portion includes a through opening and a second locking structure, the through opening has an opening length dimension and an opening width dimension, and the opening length dimension extends substantially parallel to the longitudinal axis of the tubing-securing space, and where the first locking structure is configured to extend through the through opening in the second orientation and engage the second locking structure in the first orientation, such that the tab portion is secured to the wall portion.

Another general aspect of the present disclosure includes a method of using a tubing clip, where the tubing clip is movable between a locked configuration and an unlocked configuration, and the tubing clip includes: a base portion disposed between a tab portion and a wall portion, where the tab portion includes a first locking structure, where the wall portion includes a through opening and a second locking structure, and where in the locked configuration, a tubing-securing space is formed by adjacent surfaces of the base portion, the tab portion, and the wall portion, the method including: placing a tubing on the base portion; twisting the tab portion around a longitudinal axis of the tab portion about 90 degrees to move the tab portion from the first orientation to the second orientation, where, in the unlocked configuration, the longitudinal axis of the tab portion extends substantially perpendicular to the longitudinal axis of the tubing-securing space; extending the first locking structure through the through opening while in the second orientation; allowing the tab portion to be biased back to the first orientation; and engaging the first locking structure with the second locking structure to secure the tubing in the tubing-securing space.

A device/method according to the present disclosure may include any combination of the features described above and/or the original as-filed claims.

Other systems, methods, features and advantages of the invention will be, or will become, apparent to one with skill in the art upon examination of the following figures and detailed description. It is intended that all such additional systems, methods, features and advantages be within the scope of the invention.

### BRIEF DESCRIPTION OF DRAWINGS

FIG. 1 is a perspective view of an embodiment of a tubing clip in an unlocked configuration, including a tab portion and a wall portion.

FIG. 2 is a top plan view of the tubing clip of FIG. 1.

FIG. 3 is a bottom plan view of the tubing clip of FIG. 1.

FIG. 4 is a rear elevation view of the tubing clip of FIG. 1.

FIG. 5 is a front elevation view of the tubing clip of FIG. 1.

FIG. 6 is a left side elevation view of the tubing clip of FIG. 1.

FIG. 7 is a right side elevation view of the tubing clip of FIG. 1.

FIG. 8 is a right front perspective view of the tubing clip of FIG. 1.

FIG. 9 is a left front perspective view of the tubing clip of FIG. 1.

FIG. 10 is a right rear perspective view of the tubing clip of FIG. 1.

FIG. 11 is a left bottom perspective view of the tubing clip of FIG. 1.

FIG. 12 shows a section view taken along line 12-12 of FIG. 7.

FIG. 13 is a perspective view of another embodiment of the tubing clip in a locked configuration.

FIG. 14 shows the tubing clip of FIG. 13, in a locked configuration, securing a tubing between the tab portion and the wall portion.

FIGS. 15-19 illustrate a method of moving another embodiment of the tubing clip from the unlocked configuration to the locked configuration to secure a tubing in the tubing clip.

FIG. 20 shows two tubing clips of FIG. 13, both in locked configurations, securing a tubing together.

FIG. 21 shows a system including two tubing clips of FIG. 13 affixed to an adhesive base, with a tubing secured by the system.

### DETAILED DESCRIPTION

Various embodiments are described below with reference to the drawings in which like elements generally are referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as —for example—conventional fabrication and assembly.

### Generic Description

The invention is defined by the claims, may be embodied in many different forms, and should not be construed as limited to the embodiments set forth herein; rather, these embodiments are provided so that this disclosure will be thorough and complete, and will fully convey enabling disclosure to those skilled in the art. As used in this specification and the claims, the singular forms “a,” “an,” and “the” include plural referents unless the context clearly dictates otherwise. Reference herein to any industry standards (e.g., ASTM, ANSI, IEEE standards) is defined as complying with the currently published standards as of the original filing date of this disclosure concerning the units, measurements, and testing criteria communicated by those standards unless expressly otherwise defined herein.

The terms “first,” “second,” and so on, as used herein are not meant to be assigned to a particular component so designated, but rather are simply referring to such components in the numerical order as addressed, meaning that a component designated as “first” may later be a “second” such component, depending on the order in which it is referred. It should also be understood that designation of “first” and “second” does not necessarily mean that the two components or values so designated are different, meaning for example a first scallop shaped relief structure may be the same as a second scallop shaped relief structure, with each simply being applicable to separate but identical components. The term “secure” refers to holding an object (e.g., a tube) in place so that cannot be moved laterally relative to its longitudinal axis under normal usage conditions with mild, incidental lateral forces; and, that secured object held in place by a securing item also will not move longitudinally relative to without force being applied to the secured object at a force level greater than normal usage conditions (e.g., by a user intentionally forcefully pulling the secured object while holding the securing item in place to make the former move longitudinally relative to the latter).

The terms “about,” “substantially,” “generally,” and other terms of degree, when used with reference to any volume, dimension, proportion, or other quantitative or qualitative value, are intended to communicate a definite and identifiable value within the standard parameters that would be understood by one of skill in the art (e.g., equivalent to a mechanical engineer with experience in this field), and should be interpreted to include at least any legal equivalents, minor but functionally-insignificant variants, standard manufacturing tolerances, and including at least mathematically significant figures (although not required to be as broad as the largest range thereof), including a variance of up to, for example 5%, 2%, 1%, or less or more as would be deemed appropriate by one of skill in the art. In addition, the term “configured to” is used to describe structural limitations in a particular manner that requires specific construction to accomplish a stated function and/or to interface or interact with another component(s), and is not used to describe mere intended or theoretical uses.

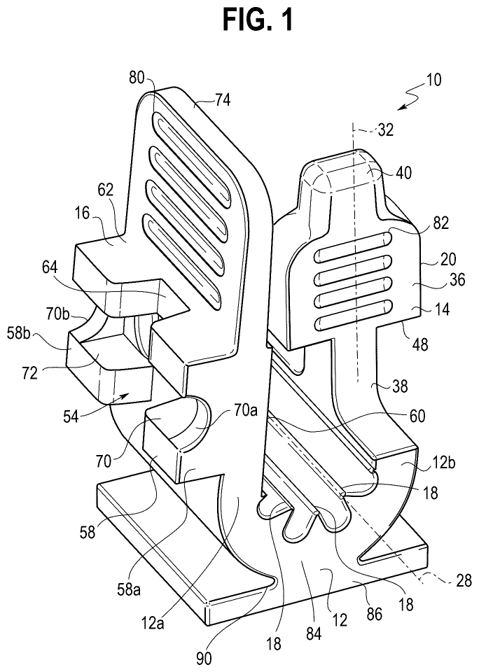

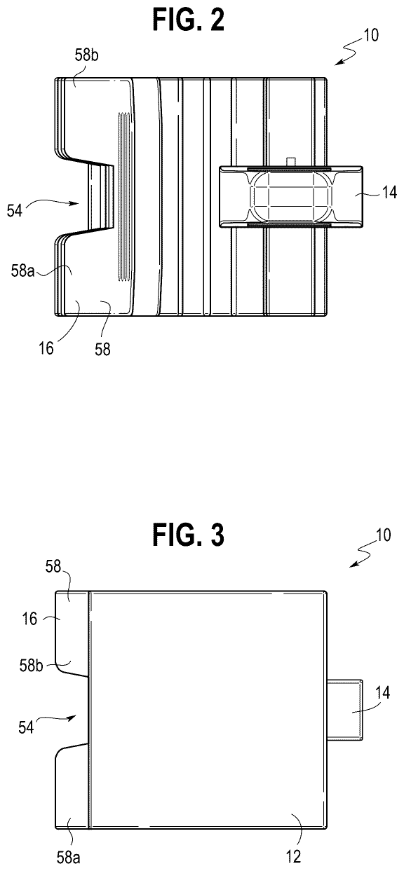

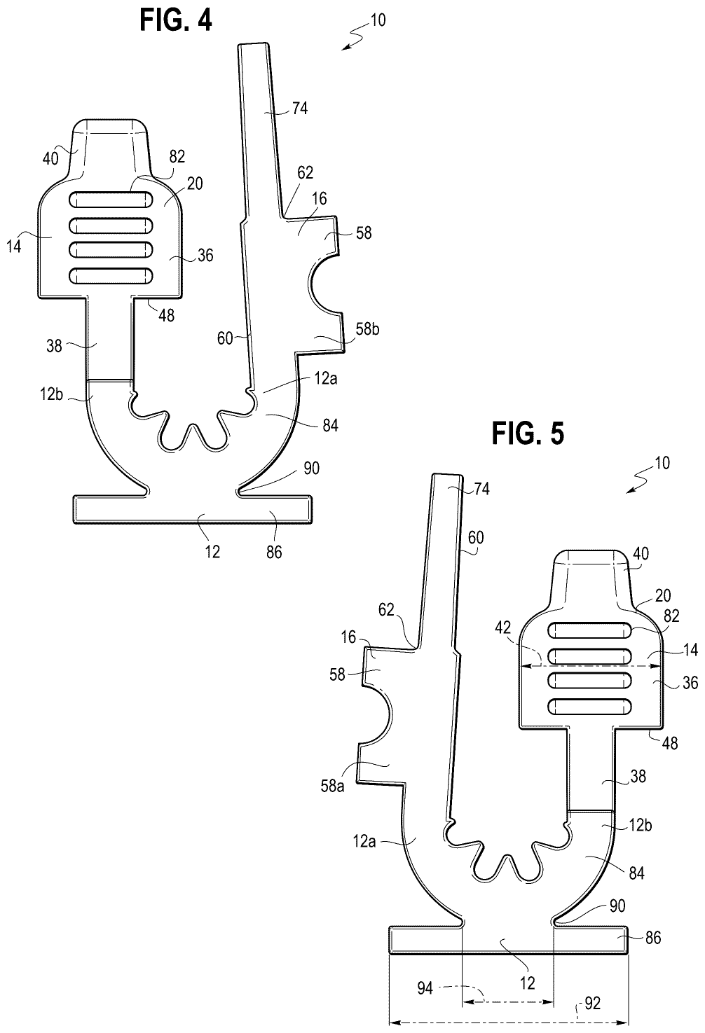

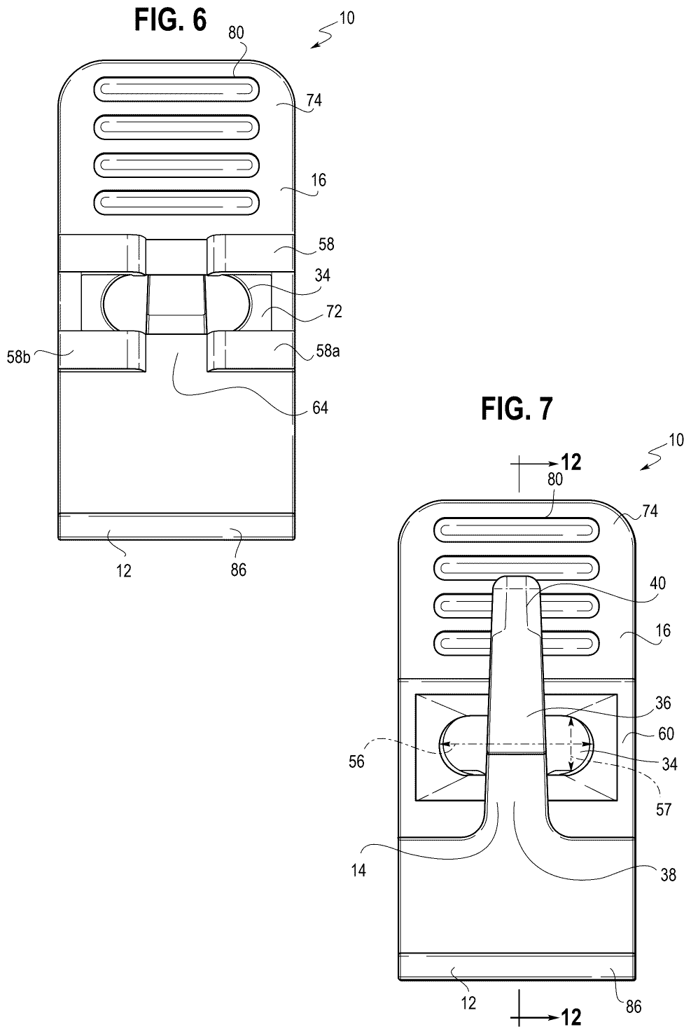

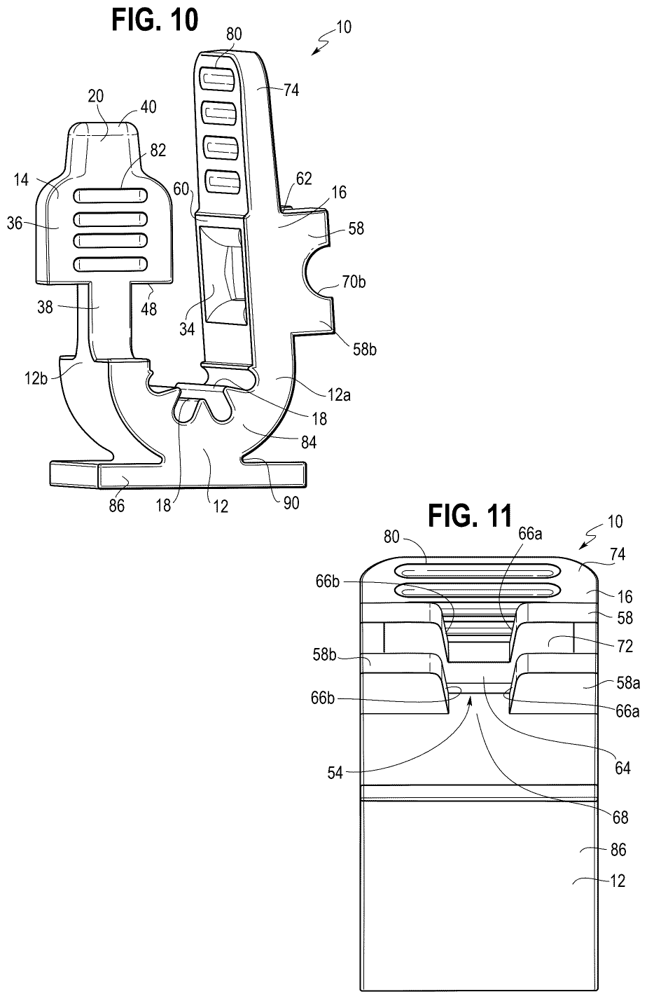

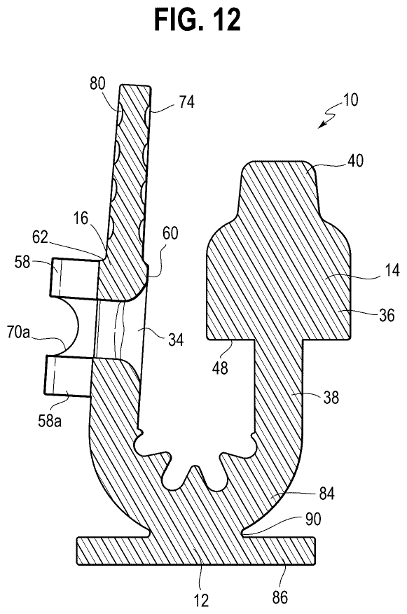

Referring to FIGS. 1-21, embodiments of a clip for securing a tubing (e.g., a tubing clip) and a method of using the clip to secure a tubing are described. For the sake of simplicity, similar features in different embodiments may be described by referring to any one or more of the embodiments, without repeating for each embodiment, but a person of ordinary skill in the art will appreciate what similar features would apply to different embodiments. Also, except for the particular surfaces and edges that must fit through/around each other, or that must contact each other or a tube to satisfy the functions described herein, the surface and edge contours, textures, and visual appearance can be varied significantly without impairing the functionality of the claimed clip embodiments.

In some embodiments, as shown in FIGS. 1-19, the clip 10 includes a base portion 12 disposed between a tab portion 14 and a wall portion 16. The base portion 12 extends between a first end 12a and a second end 12b, the wall portion 16 extends upwardly from the first end 12a of the base portion 12, and the tab portion 14 extends upwardly from the second end 12b of the base portion 12.

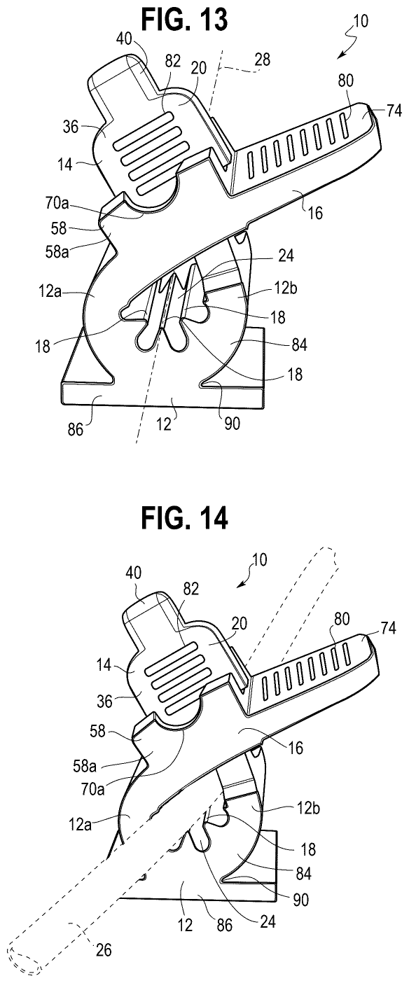

In some embodiments, as shown in FIGS. 1-19, the tab portion 14 is configured to be releasably secured to the wall portion 16 (e.g., the wall portion 16 is configured to releasably secure the tab portion 14), such that the clip 10 is movable between a locked configuration (e.g., as shown in FIGS. 13 and 14) and an unlocked configuration (e.g., as shown in FIG. 1). As shown in FIGS. 13 and 14, in the locked configuration, a space 24 (e.g., tubing-securing space 24, which may be, as shown, a generally tubular cavity) is formed among the base portion 12, the tab portion 14, and the wall portion 16, by which is meant the surfaces of those portions define a generally tubular opening that is open on one side when unlocked (e.g., as shown in FIG. 1) and generally closed when locked (e.g., as shown in FIG. 13). In other words, a tubing-securing space/opening is formed by inward facing surfaces (e.g., adjacent surfaces) of the base portion 12, the tab portion 14, and the wall portion 16.

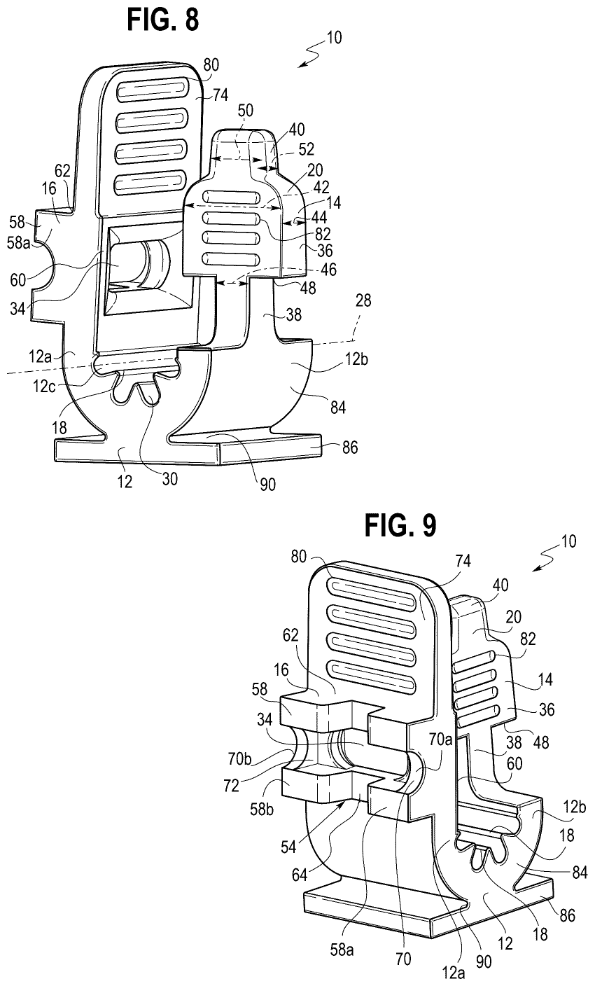

In some embodiments, as shown in FIGS. 1-19, the base portion 12 includes at least one rib 18 (which may include a plurality of ribs 18 as shown), where the at least one rib 18 protrudes into the tubing-securing space 24, and where edge(s) of the rib(s) 18 is/are configured to contact and secure at least a portion of a tubing 26 extending through the tubing-securing space 24. As shown in FIGS. 1, 8-10, and 13, the plurality of ribs 18 extend along directions that are parallel to a longitudinal axis 28 (e.g., see FIG. 13) of the tubing-securing space 24. As shown, for example in FIG. 8, an upper surface 12c of the base portion 12 includes a plurality of grooves 30 disposed on one or both sides of each rib 18 of the plurality of ribs 18. The number and the configuration (e.g., the shape, and size) of the plurality of grooves 30 may be varied, as desired and/or needed (e.g., rounded in FIG. 8) without departing from the scope of the present invention.

The clip 10 is made of a flexible material (e.g., silicone), and the tubing-securing space 24 is configured to accommodate tubes with a wide range of different diameters. Some embodiments of the clip 10 will engage tubes with diameters ranging between about 8 Fr and about 14 Fr (e.g., 8 Fr, 10 Fr, 12 Fr, and 14 Fr medical tubes), but some embodiments of the clip 10 may engage tubes with diameters ranging from about 2 Fr to much larger diameters (e.g., 34 Fr). The plurality of ribs 18 are configured to facilitate holding the tubes with different diameters in the tubing-securing space 24. This configuration of the plurality of ribs 18 and the plurality of grooves 30 are advantageous for allowing the clip 10 to be adapted to tubes with different diameters, without the need of making different clips for tubes with different diameters.

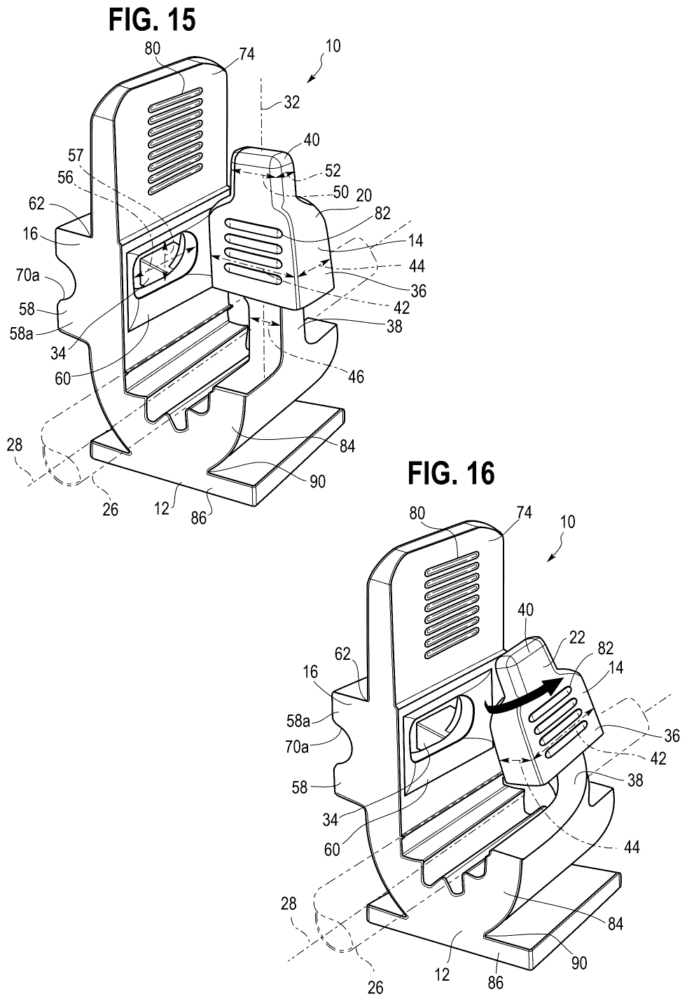

Referring to FIGS. 1-19, the tab portion 14 is resiliently biased toward a first orientation 20 (e.g., FIGS. 1 and 15) and configured to be twisted to a second orientation 22 (e.g., as shown in FIGS. 16-18), where the first orientation 20 is substantially perpendicular to the second orientation 22. “Substantially perpendicular” may include angles between 45-135 degrees, angles between 50-130 degrees, or angles between 60-120 degrees. As shown in FIG. 1, in the unlocked configuration, the longitudinal axis 32 of the tab portion 14 extends substantially perpendicular to the longitudinal axis 28 of the tubing-securing space 24. As shown in FIGS. 15 and 16, the tab portion 14 is configured to be twisted around the longitudinal axis 32 of the tab portion 14 about 90 degrees to move from the first orientation 20 to the second orientation 22.

In some embodiments, as shown in FIGS. 1 and 15, as an example, the tab portion 14 includes a first locking structure 36, a neck portion 38 disposed between the base portion 12 and the first locking structure 36, and an insertion protrusion 40 extending outwardly from the first locking structure 36. In some embodiments, as shown in FIGS. 8 and 15, for example, the first locking structure 36 has a length dimension 42 and a width dimension 44, where in the unlocked configuration and in the first orientation 20, the length dimension 42 of the first locking structure 36 extends substantially perpendicular to the longitudinal axis 28 of the tubing-securing space 24, and where in the unlocked configuration and in the second orientation 22 (e.g., as shown in FIG. 16), the length dimension 42 of the first locking structure 36 extends substantially parallel to the longitudinal axis 28 of the tubing-securing space 24.

It will be appreciated that, in the unlocked configuration, the length dimension 42 of the first locking structure 36 may extend at another angle relative to the longitudinal axis 28 of the tubing-securing space 24, without departing from the scope of the present invention, as long as the tab portion 14 may be twisted to extend through the through opening 34 and then biased back to the original orientation to engage the wall portion 16 to be locked in place. In some embodiments, the first locking structure 36 may include one or more recesses 82 or other structure(s), which add friction and facilitate grabbing the first locking structure 36, during use, thereby facilitating twisting and extending the tab portion 14.

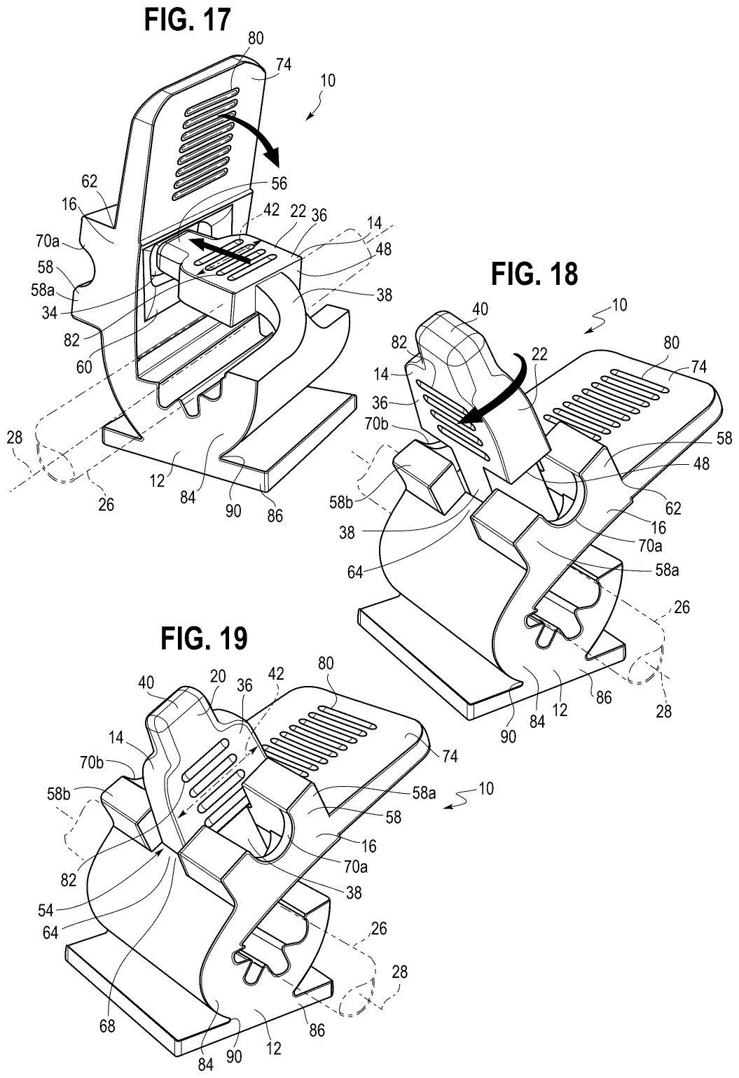

As shown in FIGS. 8 and 15, as an example, the neck portion 38 has a neck length dimension 46 that is smaller than the length dimension 42 of the first locking structure 36, and a bottom surface 48 (as best shown in FIG. 17) of the first locking structure 36 is disposed on opposite sides of the neck portion 38. The insertion protrusion 40 has an insertion length dimension 50, that is smaller than the length dimension 42 of the first locking structure 36, and an insertion width dimension 52, that is smaller than the width dimension 44 of the first locking structure 36. In use, the insertion protrusion 40 is configured to help guide the first locking structure 36 into the through opening 34.

In some embodiments, as shown in FIGS. 1 and 5, as an example, the base portion 12 includes a support portion 84 and an anchor portion 86. The support portion 84 extends outwardly from the anchor portion 86 at the connecting location 90. As shown in FIG. 1, in some embodiments, the support portion 84 extends between the first end 12a and the second end 12b and has a rounded configuration. The support portion 84 may have other non-rounded configurations, without departing from the scope of the present invention. In some embodiments, as shown in FIG. 5, as an example, the anchor portion 86 may have a length dimension 92 that is greater than a length dimension 94 of the connecting location 90. The length dimension 92 of the anchor portion 86 and the length dimension 94 of the connecting location 90 may extend in directions that are parallel to the length dimension 42 of the first locking structure 36 in the first orientation 20. In some embodiments, the anchor portion 86 extends to opposite sides of the support portion 84 and is disposed on opposite sides of the longitudinal axis 28 of the tubing-securing space 24 (e.g., as shown in FIG. 1).

Referring to FIGS. 1-19, the wall portion 16 includes a through opening 34 and a second locking structure 54. The through opening 34 extends into the wall portion 16 from a first side 60 of the wall portion 16, and the second locking structure 54 extends away from the wall portion 16 from a second side 62 of the wall portion 16. As discussed in greater detail below, the first locking structure 36 is configured to extend through the through opening 34 (in the second orientation 22, e.g., as shown in FIGS. 17 and 18, where the first locking structure 36 cannot extend through the through opening 34 in the first orientation 20) and releasably engage the second locking structure 54 of the wall portion 16 (in the first orientation 20, e.g., as shown in FIG. 19), such that the tab portion 14 is secured to the wall portion 16 and the clip 10 is in the locked configuration (when the first locking structure 36 is in the first orientation 20).

In some embodiments, as shown in FIG. 1, as an example, the wall portion 16 may include a pull portion 74 extending upwardly from an upper wall of the through opening 34. The pull portion 74 is configured to facilitate grabbing the wall portion 16 while moving the clip 10 between the unlocked configuration and the locked configuration (e.g., as shown in FIGS. 15-19). In some embodiments, the pull portion 74 may include one or more recesses 80 or other structure(s) to add friction in order to facilitate grabbing during use.

As shown in FIGS. 7 and 15, as an example, the through opening 34 has an opening length dimension 56 and an opening width dimension 57, where, in the unlocked configuration, the opening length dimension 56 extends substantially parallel to the longitudinal axis 28 of the tubing-securing space 24 and the opening width dimension 57 of the through opening 34 extends substantially perpendicular to the longitudinal axis 28 of the tubing-securing space 24.

Referring to FIGS. 15-19, the through opening 34 is configured to allow the first locking structure 36, in the second orientation 22 (e.g., as shown in FIGS. 16-18), to extend through the through opening 34. As shown in FIG. 17, the clip 10 has an intermediary configuration between the locked configuration (e.g., as shown in FIG. 19) and the unlocked configuration (e.g., as shown in FIG. 15). In the intermediary configuration, the tab portion 14 is in the second orientation 22 and the length dimension 42 of the first locking structure 36 is in registry with the opening length dimension 56 of the through opening 34.

Referring to FIG. 1, in some embodiments, the wall portion 16 includes a thickened wall structure 58 having a first portion 58a and a second portion 58b, where the first portion 58a and the second portion 58b extend away from the tab portion 14 (e.g., extending from the second side 62 of the wall portion 16), forming a locking channel 64 between the first portion 58a and the second portion 58b.

In some embodiments, as shown in FIG. 11, as an example, the locking channel 64 includes angled first sidewalls 66a of the first portion 58a and angled second sidewalls 66b of the second portion 58b, where the angled first and second sidewalls 66a and 66b lead down to a channel base 68 and they get closer as they extend towards the channel base 68. The channel base 68 is configured to engage the bottom surface 48 of the first locking structure 36 in the locked configuration (e.g., as shown in FIG. 19).

As shown in FIGS. 18 and 19, the tab portion 14 is configured to be biased to the first orientation 20 after the first locking structure 36 extending into the through opening 34 from the first side 60 of the wall portion 16 and the bottom surface 48 of the first locking structure 36 extending out of the through opening 34 from the second side 62 of the wall portion 16. The angled/tapered first and second sidewalls 66a and 66b are advantageous because they facilitate forcing the tab portion 14 to turn/snap back to the first orientation 20. The opening width dimension 57 of the through opening 34 is smaller than the length dimension 42 of the first locking structure 36, such that the first locking structure 36 is secured in the locking channel 64 in the first orientation 20 in the locked configuration. The configuration of the through opening 34, the tab portion 14, and the base portion 12 with rib(s) 18, as well as their respective orientations are advantageous, as they allow the length dimension 42 of the first locking structure 36 and at least a portion of the length of the tubing 26 secured in the clip 10 to be disposed in non-parallel directions, in the locked configuration, such that if the tubing 26 is pulled along the length of the tubing 26 or one end of the tubing 26 is pulled upward or downward relative to the base portion 12, the tab portion 14 will not disengage from the wall portion 16 accidentally.

In some embodiments, as an example, the thickened wall structure 58 includes at least one scallop shaped relief structure 70. As shown in FIG. 9, as an example, the first portion 58a of the thickened wall structure 58 has a first scallop shaped relief structure 70a and the second portion 58b of the thickened wall structure 58 has a second scallop shaped relief structure 70b, and both the first and second scallop shaped relief structures 70a and 70b have a rounded configuration (e.g., semi-circle configuration).

In some embodiments, as shown in FIG. 9, as an example, the thickened wall structure 58 also includes a cavity 72 formed between the first portion 58a and the second portion 58b, which is disposed on the second side 62 of the wall portion 16, and the cavity 72 is in registry with the through opening 34. In use, as discussed below, the cavity 72 is configured to accommodate the first locking structure 36 of the tab portion 14 in the second orientation 22 after the first locking structure 36 extends through the through opening 34 from the first side 60 of the wall portion 16. As shown in FIGS. 1 and 9, for example, the thickened wall structure 58 extends around at least a portion of the cavity 72 and the through opening 34 on the second side 62 of the wall portion 16. The first and second scallop shaped relief structures 70a and 70b are disposed on opposite sides of the cavity 72 and the through opening 34 along a direction that is parallel to the longitudinal axis 28 of the tubing-securing space 24.

The configuration of the at least one scallop shaped relief structure 70 (e.g., with a rounded configuration) is advantageous. First, it reduces the thickness of the thickened wall structure 58 around a center portion of the thickened wall structure 58, which helps the second side 62 of the wall portion 16 stretch a little more, when bended, and helps the first side 60 of the wall portion 16 bend/wrap around a tubing 26 (extending through the tubing-securing space 24) nicely, in the locked configuration of the clip 10 (e.g., as shown in FIG. 14), keeping the contact between the first side 60 and the tubing 26 in a more rounded configuration, and preventing a flat surface on the first side 60. Second, the rounded (e.g., semi-circle) configuration of the relief structure 70 also helps reduce the stress concentration on the thickened wall structure 58, in the locked configuration, by avoiding any sharp edges at the bottom of the scallop. Stretching of the rounded (e.g., semi-circle) relief structure 70 reduces the force, so that forces of the tab portion 14 against the tubing 26 do not break the wall portion 16 (e.g., around the through opening 34).

Referring to FIGS. 15-19, a method of using the clip 10 to secure a tubing will be provided below. A user may place at least a portion of a tubing 26 on the base portion 12 of the clip 10 in the unlocked configuration (e.g., as shown in FIG. 15). Then, the user may twist the tab portion 14 around the longitudinal axis 32 of the tab portion 14 (e.g., about 90 degrees) to move the tab portion 14 from the first orientation 20 to the second orientation 22 (e.g., as shown in FIG. 16). Then, the user may move the clip 10 into the intermediary configuration by bending the tab portion 14 in the second orientation 22 such that the length dimension 42 of the first locking structure 36 is in registry with the opening length dimension 56 of the through opening 34 (e.g., as shown in FIG. 17).

Then, the user may extend the first locking structure 36 through the through opening 34 while in the second orientation 22 (e.g., as shown in FIG. 18). Then, by allowing the tab portion 14 to be biased back to the first orientation 20, the first locking structure 36 will engage with the second locking structure 54, within the locking channel 64 of the second locking structure 54, to secure the tubing 26 in the tubing-securing space 24 (e.g., as shown in FIG. 19). To release the tubing 26 from the clip 10, the user may twist the tab portion 14 into the second orientation 22, and then retract the first locking structure 36 through the through opening 34, thereby disengaging the first locking structure 36 from the second locking structure 54, such that the clip 10 is moved from the locked configuration to the unlocked configuration, allowing the tubing 26 to be removed from the base portion 12 of the clip 10.

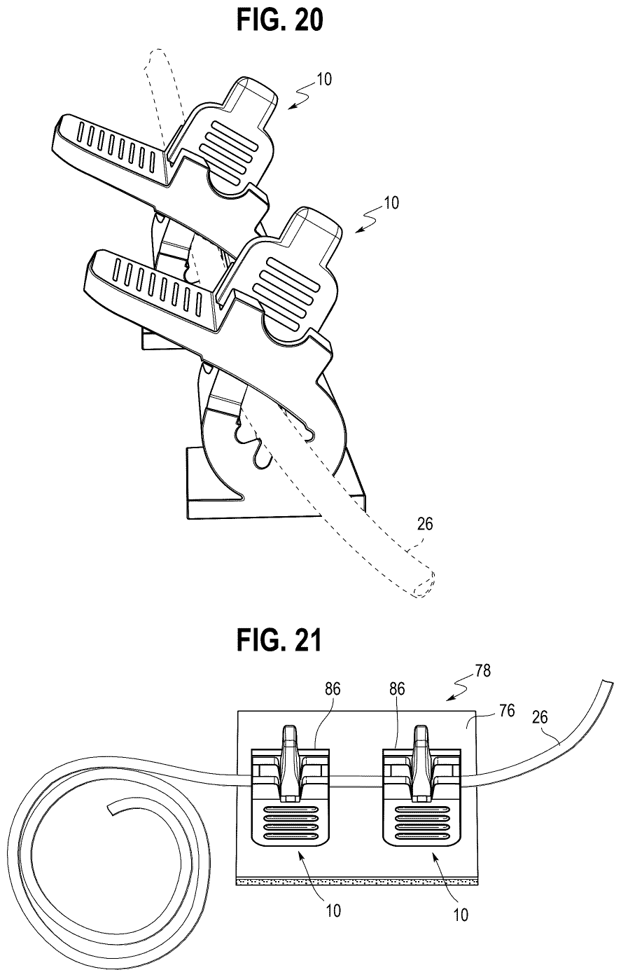

Referring to FIG. 20, as an example, two or more clips 10 may be used together to secure a tubing 26. In some embodiments, as shown in FIG. 21, a system 78 may be provided with a plurality of clips 10, where the plurality of clips 10 is affixed to one or more adhesive bases 76. For example, in medical procedures, the user may secure the one or more adhesive bases 76 first, on a person's skin or other places, as desired and/or needed, and then after the medical tubing 26 is placed at a desired location, the user may use the system 78 to secure the medical tubing 26 in place, according to the method discussed above. In some embodiments, as shown in FIG. 21, as an example, the anchor portion 86 is configured to provide a surface area for adhesion, capture, or other engagement or attachment with the one or more adhesive bases 76. The one or more adhesive bases 76 may be affixed to any surface (e.g., the top surface, the bottom surface) of the anchor portion 86 without departing from the scope of the present invention.

The subject matter of the disclosure may also relate, among others, to the following aspects:

A first aspect relates to a clip for securing a tubing, comprising: a base portion disposed between a tab portion and a wall portion, wherein the base portion includes at least one rib, wherein the tab portion is resiliently biased toward a first orientation and configured to be twisted to a second orientation, wherein the wall portion is configured to releasably secure the tab portion, wherein the clip is movable between a locked configuration and an unlocked configuration, wherein the tab portion includes a first locking structure, the wall portion includes a through opening and a second locking structure, and the first locking structure is configured to extend through the through opening when in the second orientation (but not in the first orientation) and to releasably engage the second locking structure of the wall portion, such that the tab portion is secured to the wall portion and the clip is in the locked configuration when in the first orientation, and wherein, in the locked configuration, a tubing-securing space is formed among the base portion, the tab portion, and the wall portion, and the at least one rib protrudes into the tubing-securing space.

A second aspect relates to the clip of aspect 1, wherein the first orientation is substantially perpendicular to the second orientation.

A third aspect relates to the clip of any one of aspects 1 or 2, wherein the first locking structure has a length dimension and a width dimension, wherein in the unlocked configuration and in the first orientation, the length dimension of the first locking structure extends substantially perpendicular to a longitudinal axis of the tubing-securing space, and wherein in the unlocked configuration and in the second orientation, the length dimension of the first locking structure extends substantially parallel to the longitudinal axis of the tubing-securing space.

A fourth aspect relates to the clip of any preceding aspect, wherein the through opening has an opening length dimension and an opening width dimension, wherein, in the unlocked configuration, the opening length dimension extends substantially parallel to the longitudinal axis of the tubing-securing space and the opening width dimension of the through opening extends substantially perpendicular to the longitudinal axis of the tubing-securing space, wherein the opening width dimension is smaller than the length dimension of the first locking structure, and wherein the through opening is configured to allow the first locking structure, in the second orientation, to extend through the through opening.

A fifth aspect relates to the clip of any preceding aspect, wherein the tab portion further includes a neck portion disposed between the base portion and the first locking structure, wherein the neck portion has a neck length dimension that is smaller than the length dimension of the first locking structure, and a bottom surface of the first locking structure is disposed on opposite sides of the neck portion, and wherein the tab portion is configured to be biased to the first orientation after the first locking structure extending into the through opening from a first side of the wall portion and the bottom surface of the first locking structure extending out of the through opening from a second side of the wall portion.

A sixth aspect relates to the clip of any preceding aspect, wherein the tab portion further includes an insertion protrusion extending outwardly from the first locking structure, wherein the insertion protrusion has an insertion length dimension, that is smaller than the length dimension of the first locking structure, and an insertion width dimension, that is smaller than the width dimension of the first locking structure.

A seventh aspect relates to the clip of any preceding aspect, wherein the wall portion further includes a thickened wall structure having a first portion and a second portion, wherein the first portion and the second portion extend away from the tab portion, forming a locking channel between the first portion and the second portion.

An eighth aspect relates to the clip of any preceding aspect, wherein the locking channel includes angled sidewalls leading down to a channel base, and wherein the channel base is configured to engage a bottom surface of the first locking structure in the locked configuration.

A ninth aspect relates to the clip of any preceding aspect, wherein the thickened wall structure includes at least one scallop shaped relief structure.

A tenth aspect relates to the clip of any preceding aspect, wherein the scallop shaped relief structure has a rounded configuration.

An eleventh aspect relates to the clip of any preceding aspect, wherein the base portion includes a support portion and an anchor portion, the support portion extends outwardly from the anchor portion at a connecting location, and the anchor portion has a length dimension that is greater than a length dimension of the connecting location.

A twelfth aspect relates to a system comprising a plurality of clips of any preceding aspect, wherein the plurality of clips is affixed to one or more adhesive bases.

A thirteenth aspect relates to the clip of any preceding aspect, wherein the base portion includes a support portion and an anchor portion, the support portion extends outwardly from the anchor portion at a connecting location, and the anchor portion has a length dimension that is greater than a length dimension of the connecting location, and wherein the plurality of clips is affixed to the one or more adhesive bases via the anchor portion of each clip of the plurality of clips.

A fourteenth aspect relates to a clip for securing a tubing, comprising: a base portion extending between a first end and a second end; a wall portion extending upwardly from the first end of the base portion; a tab portion extending upwardly from the second end of the base portion, wherein the tab portion is resiliently biased toward a first orientation and configured to be twisted to a second orientation, wherein the clip is movable between a locked configuration and an unlocked configuration, and in the locked configuration, a tubing-securing opening is formed by inward facing surfaces of the base portion, the tab portion, and the wall portion, wherein the tab portion includes a first locking structure, and the first locking structure has a length dimension and a width dimension, wherein in the unlocked configuration and in the first orientation, the length dimension of the first locking structure extends substantially perpendicular to a longitudinal axis of the tubing-securing space, wherein in the unlocked configuration and in the second orientation, the length dimension of the first locking structure extends substantially parallel to the longitudinal axis of the tubing-securing space, wherein the wall portion includes a through opening and a second locking structure, the through opening has an opening length dimension and an opening width dimension, and the opening length dimension extends substantially parallel to the longitudinal axis of the tubing-securing space, and wherein the first locking structure is configured to extend through the through opening in the second orientation and engage the second locking structure in the first orientation, such that the tab portion is secured to the wall portion.

A fifteenth aspect relates to the clip of aspect 14, wherein the clip has an intermediary configuration, and wherein in the intermediary configuration, the tab portion is in the second orientation and the length dimension of the first locking structure is in registry with the opening length dimension of the through opening.

A sixteenth aspect relates to the clip of any one of aspects 14 or 15, wherein the wall portion includes a pull portion extending upwardly from an upper wall of the through opening.

A seventeenth aspect relates to the clip of any one of aspects 14 to 16, wherein the tab portion further includes a neck portion disposed between the base portion and the first locking structure, wherein the neck portion has a neck length dimension that is smaller than the length dimension of the first locking structure, and a bottom surface of the first locking structure is disposed on opposite sides of the neck portion, wherein the wall portion includes a thickened wall structure having a first portion and a second portion, wherein the first portion and the second portion extend away from the tab portion, forming a locking channel between the first portion and the second portion, and wherein, in the locked configuration, the first locking structure is secured in the locking channel in the first orientation.

An eighteenth aspect relates to the clip of any one of aspects 14 to 17, wherein, in the unlocked configuration, the tab portion is configured to be twisted around a longitudinal axis of the tab portion about 90 degrees to move from the first orientation to the second orientation, and wherein, in the unlocked configuration, the longitudinal axis of the tab portion extends substantially perpendicular to the longitudinal axis of the tubing-securing space.

A nineteenth aspect relates to a method of using a tubing clip, wherein the tubing clip is movable between a locked configuration and an unlocked configuration, and the tubing clip comprises: a base portion disposed between a tab portion and a wall portion, wherein the tab portion includes a first locking structure, wherein the wall portion includes a through opening and a second locking structure, and wherein in the locked configuration, a tubing-securing space is formed by adjacent surfaces of the base portion, the tab portion, and the wall portion, the method comprising: placing a tubing on the base portion; twisting the tab portion around a longitudinal axis of the tab portion about 90 degrees to move the tab portion from the first orientation to the second orientation, wherein, in the unlocked configuration, the longitudinal axis of the tab portion extends substantially perpendicular to the longitudinal axis of the tubing-securing space; extending the first locking structure through the through opening while in the second orientation; allowing the tab portion to be biased back to the first orientation; and engaging the first locking structure with the second locking structure to secure the tubing in the tubing-securing space.

A twentieth aspect relates to the method of aspect 19, further comprising: twisting the tab portion into the second orientation; retracting the first locking structure through the through opening; and disengaging the first locking structure from the second locking structure.

A twenty-first aspect relates to the method of any one of aspects 19 or 20, further comprising: engaging the first locking structure within a locking channel of the second locking structure.

A twenty-second aspect relates to the method of any one of aspects 19 to 21, wherein the clip has an intermediary configuration between the locked configuration and the unlocked configuration, wherein the through opening has an opening length dimension and an opening width dimension, wherein, in the unlocked configuration, the opening length dimension extends substantially parallel to the longitudinal axis of the tubing-securing space and the opening width dimension of the through opening extends substantially perpendicular to the longitudinal axis of the tubing-securing space, wherein the first locking structure has a length dimension and a width dimension, and wherein in the unlocked configuration and in the first orientation, the length dimension of the first locking structure extends substantially perpendicular to the longitudinal axis of the tubing-securing space, the method further comprising: moving the clip into the intermediary configuration by bending the tab portion in the second orientation such that the length dimension of the first locking structure is in registry with the opening length dimension of the through opening.

Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation unless specifically defined by context, usage, or other explicit designation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment. In the event of any inconsistent disclosure or definition from the present application conflicting with any document incorporated by reference, the disclosure or definition herein shall be deemed to prevail.

We claim:

1. A clip for securing a tubing, comprising:
a base portion disposed between a tab portion and a wall portion,
wherein the base portion includes at least one rib,
wherein the tab portion is resiliently biased toward a first orientation and configured to be twisted to a second orientation,
wherein the wall portion is configured to releasably secure the tab portion,
wherein the clip is movable between a locked configuration and an unlocked configuration,
wherein the tab portion includes a first locking structure, the wall portion includes a through opening and a second locking structure, and the first locking structure is configured to extend through the through opening when in the second orientation (but not in the first orientation) and to releasably engage the second locking structure of the wall portion, such that the tab portion is secured to the wall portion and the clip is in the locked configuration when in the first orientation, and
wherein, in the locked configuration, a tubing-securing space is formed among the base portion, the tab portion, and the wall portion, and the at least one rib protrudes into the tubing-securing space.

2. The clip of claim 1, wherein the first orientation is substantially perpendicular to the second orientation.

3. The clip of claim 1, wherein the first locking structure has a length dimension and a width dimension, wherein in the unlocked configuration and in the first orientation, the length dimension of the first locking structure extends substantially perpendicular to a longitudinal axis of the tubing-securing space, and wherein in the unlocked configuration and in the second orientation, the length dimension of the first locking structure extends substantially parallel to the longitudinal axis of the tubing-securing space.

4. The clip of claim 3, wherein the through opening has an opening length dimension and an opening width dimension, wherein, in the unlocked configuration, the opening length dimension extends substantially parallel to the longitudinal axis of the tubing-securing space and the opening width dimension of the through opening extends substantially perpendicular to the longitudinal axis of the tubing-securing space, wherein the opening width dimension is smaller than the length dimension of the first locking structure, and wherein the through opening is configured to allow the first locking structure, in the second orientation, to extend through the through opening.

5. The clip of claim 4, wherein the tab portion further includes a neck portion disposed between the base portion and the first locking structure, wherein the neck portion has a neck length dimension that is smaller than the length dimension of the first locking structure, and a bottom surface of the first locking structure is disposed on opposite sides of the neck portion, and wherein the tab portion is configured to be biased to the first orientation after the first locking structure extending into the through opening from a first side of the wall portion and the bottom surface of the first locking structure extending out of the through opening from a second side of the wall portion.

6. The clip of claim 3, wherein the tab portion further includes an insertion protrusion extending outwardly from the first locking structure, wherein the insertion protrusion has an insertion length dimension, that is smaller than the length dimension of the first locking structure, and an insertion width dimension, that is smaller than the width dimension of the first locking structure.

7. The clip of claim 1, wherein the wall portion further includes a thickened wall structure having a first portion and a second portion, wherein the first portion and the second portion extend away from the tab portion, forming a locking channel between the first portion and the second portion.

8. The clip of claim 7, wherein the locking channel includes angled sidewalls leading down to a channel base, and wherein the channel base is configured to engage a bottom surface of the first locking structure in the locked configuration.

9. The clip of claim 7, wherein the thickened wall structure includes at least one scallop shaped relief structure.

10. The clip of claim 9, wherein the scallop shaped relief structure has a rounded configuration.

11. The clip of claim 1, wherein the base portion includes a support portion and an anchor portion, the support portion extends outwardly from the anchor portion at a connecting location, and the anchor portion has a length dimension that is greater than a length dimension of the connecting location.

12. A system comprising a plurality of clips according to claim 1, wherein the plurality of clips is affixed to one or more adhesive bases.

13. The system of claim 12, wherein the base portion includes a support portion and an anchor portion, the support portion extends outwardly from the anchor portion at a connecting location, and the anchor portion has a length dimension that is greater than a length dimension of the connecting location, and wherein the plurality of clips is affixed to the one or more adhesive bases via the anchor portion of each clip of the plurality of clips.

14. A clip for securing a tubing, comprising:
a base portion extending between a first end and a second end;
a wall portion extending upwardly from the first end of the base portion;
a tab portion extending upwardly from the second end of the base portion, wherein the tab portion is resiliently biased toward a first orientation and configured to be twisted to a second orientation,
wherein the clip is movable between a locked configuration and an unlocked configuration, and in the locked configuration, a tubing-securing opening is formed by inward facing surfaces of the base portion, the tab portion, and the wall portion,
wherein the tab portion includes a first locking structure, and the first locking structure has a length dimension and a width dimension,
wherein in the unlocked configuration and in the first orientation, the length dimension of the first locking structure extends substantially perpendicular to a longitudinal axis of the tubing-securing space,
wherein in the unlocked configuration and in the second orientation, the length dimension of the first locking structure extends substantially parallel to the longitudinal axis of the tubing-securing space,
wherein the wall portion includes a through opening and a second locking structure, the through opening has an opening length dimension and an opening width dimension, and the opening length dimension extends substantially parallel to the longitudinal axis of the tubing-securing space, and
wherein the first locking structure is configured to extend through the through opening in the second orientation and engage the second locking structure in the first orientation, such that the tab portion is secured to the wall portion.

15. The clip of claim 14, wherein the clip has an intermediary configuration, and wherein in the intermediary configuration, the tab portion is in the second orientation and the length dimension of the first locking structure is in registry with the opening length dimension of the through opening.

16. The clip of claim 14, wherein the wall portion includes a pull portion extending upwardly from an upper wall of the through opening.

17. The clip of claim 14, wherein the tab portion further includes a neck portion disposed between the base portion and the first locking structure, wherein the neck portion has a neck length dimension that is smaller than the length dimension of the first locking structure, and a bottom surface of the first locking structure is disposed on opposite sides of the neck portion, wherein the wall portion includes a thickened wall structure having a first portion and a second portion, wherein the first portion and the second portion extend away from the tab portion, forming a locking channel between the first portion and the second portion, and wherein, in the locked configuration, the first locking structure is secured in the locking channel in the first orientation.

18. The clip of claim 14, wherein, in the unlocked configuration, the tab portion is configured to be twisted around a longitudinal axis of the tab portion about 90 degrees to move from the first orientation to the second orientation, and wherein, in the unlocked configuration, the longitudinal axis of the tab portion extends substantially perpendicular to the longitudinal axis of the tubing-securing space.

19. A method of using a tubing clip, wherein the tubing clip is movable between a locked configuration and an unlocked configuration, and the tubing clip comprises:
a base portion disposed between a tab portion and a wall portion,
wherein the tab portion includes a first locking structure,
wherein the wall portion includes a through opening and a second locking structure, and
wherein in the locked configuration, a tubing-securing space is formed by adjacent surfaces of the base portion, the tab portion, and the wall portion,
the method comprising:
placing a tubing on the base portion;
twisting the tab portion around a longitudinal axis of the tab portion about 90 degrees to move the tab portion from the first orientation to the second orientation, wherein, in the unlocked configuration, the longitudinal axis of the tab portion extends substantially perpendicular to the longitudinal axis of the tubing-securing space;
extending the first locking structure through the through opening while in the second orientation;
allowing the tab portion to be biased back to the first orientation; and
engaging the first locking structure with the second locking structure to secure the tubing in the tubing-securing space.

20. The method of claim 19, further comprising:
twisting the tab portion into the second orientation;
retracting the first locking structure through the through opening; and
disengaging the first locking structure from the second locking structure.

21. The method of claim 19, further comprising: engaging the first locking structure within a locking channel of the second locking structure.

22. The method of claim 19, wherein the clip has an intermediary configuration between the locked configuration and the unlocked configuration, wherein the through opening has an opening length dimension and an opening width dimension, wherein, in the unlocked configuration, the opening length dimension extends substantially parallel to the longitudinal axis of the tubing-securing space and the opening width dimension of the through opening extends substantially perpendicular to the longitudinal axis of the tubing-securing space, wherein the first locking structure has a length dimension and a width dimension, and wherein in the unlocked configuration and in the first orientation, the length dimension of the first locking structure extends substantially perpendicular to the longitudinal axis of the tubing-securing space, the method further comprising:
moving the clip into the intermediary configuration by bending the tab portion in the second orientation such that the length dimension of the first locking structure is in registry with the opening length dimension of the through opening.
